(19) United States
(12) Patent Application Publication
(10) Pub. No.: US 2006/0269939 A1
(43) Pub. Date: Nov. 30, 2006

(54) Method for conversion of a DNA sequence to a number string and applications thereof in the field of accelerated drug design

(75) Inventors: Vivek Kumar Singh, Naini Allahabad (U.P.) (IN); Vivek Gangadhar Mahale, Nashik (IN); Avinash Purshottam Agnihotry, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignee: MASCON GLOBAL LIMITED, New Delhi (IN)

(21) Appl. No.: 11/403,323

(22) Filed: Apr. 13, 2006

(30) Foreign Application Priority Data
Apr. 15, 2005 (IN) 953/DEL/2005

(51) Int. Cl. C12Q 1/68; G06F 19/00

(52) U.S. Cl. 435/6; 702/20

(57) ABSTRACT

The present invention relates to a method for the conversion of a DNA sequence into a number string. More particularly, the present invention relates to a method for the conversion of a DNA sequence into a number string using a genomic numbering system in order to extract and/or analyze biological information. The method of the invention is particularly useful in the development of new drugs or active chemical agents.

### FIELD OF THE INVENTION

The present invention relates to a method for the conversion of a DNA sequence into a number string. More particularly, the present invention relates to a method for the conversion of a DNA sequence into a number string using a genomic numbering system in order to extract and/or analyze biological information. The method of the invention is particularly useful in the development of new drugs or active chemical agents.

### BACKGROUND OF THE INVENTION

DNA is an excellent molecular electronic device since it stores, processes and provides information for growth and maintenance of living systems. All living species are a result of a single cell produced during reproduction. In most of the cases this single cell does not have most of the materials required for fabricating a living system but contains all the information and processing capability to fabricate living spaces by taking materials from environment, for example, fabrication of baby from Zygote which contains rearranged DNA sequences of parents. DNA is a ready to use nanowire of 2 nm and can be synthesized in any sequence of four bases i.e. A,T,G,C. DNA of every living organism (micro/macro) consist of large number of DNA segments where each segment represents a processor to execute a particular biological process for growth and maintaining life.

Clelland et al., 1999 (Hiding messages in DNA microdots. Nature. 399, 533-534 (1999), and Bancroft, et al. 2001 [U.S. Pat. No. 6,312,911], have developed a DNA based steganographic technique for sending secret messages. Although their prime objective was steganography (the art of information hiding), they used DNA as a storage and transmission device for secret message. They encrypted the plaintext message into the DNA sequences and retrieved the message using the encryption/decryption key. The important feature of this disclosure is that they used three DNA bases for representing a single alphanumeric character. The focus of the numbering system followed therein was towards storage and transmission of encrypted data via DNA.

A gene is the stretch of DNA that can be coded for functional product (e.g. Protein, RNA), which is the material for fabrication. A significant problem is that of deducing the amino acid sequences encoded in a given DNA genomic sequence in order to understand the expression of genes in a genome. In prokaryotes gene identification is easier since the coding regions are small continuous strings of DNA. However, in the case of higher eukaryotic organisms, genes are often split in a number of coding fragments known as exons, separated by non-coding intervening fragments known as introns.

Gene identification is essentially effected using both intrinsic information derived from the query sequence itself which could be signal based or content based, as well as extrinsic information by comparing the query sequence with other known sequences in public databases. Examples of sequence signals are promoters, splice sites, CpG islands etc. and a wide variety of methods exist to score and locate sequence signals for gene identification. Content refers to information derived from the fact that coding regions in the DNA exhibit peculiar sequence statistical properties. In the case of extrinsic information, since all genomes are interrelated, the existence of homologous sequences can both validate a gene prediction as well as give some idea of gene function. In addition to coding regions, Expressed Sequence Tags (ESTs) can also reveal function, but homology at the level of promoters, or even intrinsically non-coding sequences, such as repeats have been explored for useful information.

A coding statistic assists in determination of a real number for a given DNA sequence, and which is related to the likelihood that the particular sequence is coding for a protein. Although in practice the values of a given coding statistic can be computed in a number of ways but these can be broadly categorized into measures that depend on coding DNA and measures that are independent of coding DNA. Model dependent statistics are likely to capture more of the specific features of coding DNA whereas model independent statistics capture only the “universal” features of coding DNA; since they do not require of a sample of coding DNA, they can be used even in absence of previously known coding regions from the species under consideration. The former are knowledge-based methods while the latter are ab-initio techniques.

Knowledge based methods include measures based on oligonucleotide count like codon usage, amino acid usage codon preference, hexamer usage, measures based on composition bias between codon position i.e. codon prototype, measures based on dependence between nucleotide position e.g. Markov models and hidden Markov models (HMM).

Unequal usage of codon in the coding regions appears to be a universal feature of the genomes across the phylogenetic spectra. This bias obeys mainly to
1) The uneven usage of amino acids in existing proteins
2) The uneven usage of synonymous codons. Bias in the distribution of oligonucleotide other than codons (trinucleotide) can also be used to discriminate between coding and non-coding regions. Bias in the usage of hexamers may be the most discriminant one (probably because of dependence between adjacent amino acids in the proteins).

In cases where only small fraction of total possible genes are known, non-biased methods are required which do not require a training set. Such ab initio methods include measures based on base compositional bias between codon positions. In such methods the asymmetric distribution of nucleotides at three triplets' positions in the sequence is measured. Alternatively, measures based on periodic correlation between nucleotide positions where a number of coding statistics have been devised based in measuring the periodic structure or the co-relational structure of DNA sequences can also be used.

Periodic Asymmetry Index (PAI) can be used to measure the tendency to cluster homogenous di-nucleotides in a three base periodic pattern can be measured by the PAI. Average Mutual Information (AMI) can be used to compute how many types of nucleotide I is followed by a nucleotide J at a distance K in a given DNA sequence.

Other prior art methods include measurement of Fourier Spectrum. Fourier analysis permits and enables periodic correlation in DNA sequences. DNA coding regions reveal the characteristic periodicity of 3 as a distinct peak at frequency f=1/3. TIWARI, S., RAMACHANDRAN, S., BHATTACHARYA, A., BHATTACHARYA, S., AND RAMASWAMY, R. 1997. Prediction of probable genes by fourier analysis of genomic sequences. Computer Applications in the Biosciences 13:263-270.

Fourier Transform Mass Spectrometry (FTMS) is also known as Fourier Transform Ion Cyclotron Resonance (FTICR). The principle of molecular mass determination used in FTMS is based on a linear relationship between an ion's mass and its cyclotron frequency. In a uniform magnetic field, an ion will process about the center of the magnetic field in a periodic, circular motion known as cyclotron motion. An ensemble of ions having a particular mass-to-charge ratio (m/z) can be made to undergo cyclotron motion in-phase, producing an image current. The image current is detected between a pair of receive electrodes, producing a sine-wave signal. The Fourier transform is a mathematical deconvolution method used to separate the signals from many different m/z ensembles into a frequency, also known as mass spectrum.

The prior art methods suffer from several disadvantages, which are enumerated below. The methods using hidden Markov models use training based system, which therefore requires training to identify genes. Such methods are organisms or dataset specific and cannot be applied to newly sequenced genomes or organisms where the information available is limited. This affects the accuracy. The result obtained is biased since it is dataset dependent. Methods using ANN also suffer from the same disadvantages as the hidden Markov Model systems.

Fourier spectrum based methods are ab initio based and use intrinsic properties of the sequence to find the coding region. The method uses linear mapping to convert the DNA to signal, whereas genome is nonlinear in nature.

The DNA walk based systems are also ab initio based and use the periodic correlation between nucleotide positions of sequence to find coding region. The method projects a global behavior whereas short range interaction is not a factor.

Integrated methods which combine homology information use various algorithms to increase their accuracy by drawing homology information from different databases.

While much progress has been made in recent years in traditional molecular and genetic mapping, sequencing of genomes and molecular analysis of gene expression, there is still a tremendous need to develop improved techniques for molecular and genetic analysis within and between species.

Molecular markers are common tools that can reveal polymorphism directly at the DNA level and are used for genetic resource assessment, molecular analysis and genetic mapping. Various types of markers have been developed.

RFLP: Restriction Fragment Length Polymorphism.

PCR: Polymerase Chain Reaction based markers.

SCAR: Sequence Characterized Amplified Region.

SSR: Simple Sequence Repeats (micro satellites).

ISSR: Inter Simple Sequence Repeats.

STS: Sequence Tagged Sites.

AFLP: Amplified Fragment Length Polymorphisms.

Although these methods are powerful, they are useful only within one species or genus because the markers are not from genes shared by larger taxonomic groups. There is thus a need in the art to develop improved methods of genetic mapping and molecular analysis within and across different kingdoms.

It is accepted that gene identification is a crucial step in the development of new drugs. Conventional processes of genome to drugs proceed using the following steps:
(a) finding all genes from the host and the target;
(b) finding important enzymes which are unique to the target organism;
(c) subjecting the genes and enzymes to protein-protein interaction studies.

It is important to reduce the cost and time taken in drug designing. The method of the invention results in cost and time saving in drug designing by reducing the number of false negative and false positive genes. The protein—protein interaction study uses comparison of two different proteins at the level of their genomic numbering, thereby simplifying the method of gene identification and drug development.

Advances in techniques for sequencing long stretches of genomic deoxyribonucleic acid (DNA) have allowed investigators to collect vast nucleic acid sequence data rapidly. These advances, combined with initiatives to sequence the entire human genome and the genomes of several other species, have created a need for the rapid identification of genes on long stretches of sequenced DNA. Conventional gene location techniques, such as cDNA hybridization, are effective at locating transcribed genes, but are time-consuming and costly, thereby increasing the cost and time for development of new drug.

An alternative for locating genes on DNA that has not otherwise been analyzed for potential coding regions involves using statistical detection methods. Such methods conventionally include using probability models to predict where in a DNA sequence a gene is located. The theoretical nucleic acid sequence probabilities can be determined through analysis of known coding regions in the organism of interest. Once theoretical nucleic acid sequence probabilities are determined, nucleic acid sequences in non-annotated regions of DNA in the same or a similar organism can be statistically compared to the theoretical nucleic acid sequence probabilities. If the similarity is sufficient, the investigator is notified that a coding sequence exists. Conventional cloning techniques can then be used to isolate the putative gene and check for transcription.

One type of statistical detection method searches DNA by content. In such content-based models, highly conserved regions of DNA that are common to all genes are located. If a conserved region of DNA is found, then the nucleic acid sequence associated with the conserved region can be compared with known genes. Such comparisons, which can be done with nucleic acid sequence comparison programs such as BLAST, works if similar nucleotide or protein sequence is present, content-based searches therefore have limited desirability as they through lot of false positives there by increasing the processing. These types of methods fail to detect a novel gene, which has no homologous in the Database.

A second type of statistical detection method searches DNA by signal. This type of searching involves using probability models to predict whether DNA fragments within a larger nucleic acid sequence are coding. Early searching by signal programs, such as Test Code and Grail, relied on statistical variations within coding regions of DNA, including codon frequency, local nucleic acid sequence composition, codon preference measures, heuristics based on oligonucleotide frequency variations, and measures of nucleic acid sequence complexity.

Beyond simple gene detection, there is also a need for the determination of other coding features, such as the location of intron/exon boundaries in eukaryotic organisms and the location of insertions or deletions. The program GENSCAN (Burge, C. and Karlin, S. (1997) Prediction of Complete Gene Structures in Human Genomic DNA. J. Mol. Biol. 268, 78-94), for example, predicts exon location with local state probabilities based on oligonucleotide usage. GENSCAN, however, also depends on non-local nucleic acid sequence characteristics, which make the program very sensitive to sequencing errors and genes containing alternative splicing strategies.

One statistical model that avoids the problems caused by dependence on non-local nucleic acid sequence characteristics is the inhomogeneous Markov model. An inhomogeneous Markov model depends upon local probabilities, and is not therefore sensitive to sequencing errors or genes with alternative splicing strategies. The inhomogeneous Markov model is “inhomogeneous” because it determines the state probabilities for a given nucleotide in multiple reading frames rather than in a single reading frame. GeneMark, for example, is a computer program that uses the inhomogeneous Markov model to locate genes.

The GeneMark gene prediction algorithm was developed in several steps. A series of three publications demonstrated that inhomogeneous Markov models were useful tools for gene prediction (see Borodovsky, M., Sprizhitsky Yu., Golovanov E. and Alexandrov A. (1986).

Borodovsky, M., Sprizhitsky Yu., Golovanov E. and Alexandrov A. (1986) Statistical Patterns in Primary Structures of Functional Regions in the E. Coli Genome: I. Oligonucleotide Frequencies Analysis, Molecular Biology, 20, 826-833.

Statistical Patterns in Primary Structures of Functional Regions in the E. Coli Genome: I. Oligonucleotide Frequencies Analysis, Molecular Biology, 20, 826-833, Borodovsky, M., Sprizhitsky Yu, Golovanov E. and Alexandrov A. (1986) Statistical Patterns in Primary Structures of Functional Regions in the E. Coli Genome: II. Non-homogeneous Markov Models, Molecular Biology, 20, 833-840, Borodovsky, M., Sprizhitsky Yu., Golovanov E. and Alexandrov A. (1986) Statistical Patterns in Primary Structures of Functional Regions in the E. Coli Genome: III. Computer Recognition of Coding Regions, Molecular Biology, 20, 1145-1150, all of which are herein incorporated by reference in their entirety). The GeneMark method was based on an inhomogeneous Markov model and was described in 1993 (see Borodovsky, M. and McIninch J. (1993) GeneMark, Parallel Gene Recognition for both DNA Strands, Computers & Chemistry, 17, 123-133, and Borodovsky, M. and McIninch J. (1993) BioSystems v30, pp. 161-171, both of which are herein incorporated by reference in their entirety). The capabilities of the GeneMark program were subsequently investigated (see James D. McIninch, Prediction of Protein Coding Regions in Unannotated DNA sequences Using an Inhomogeneous Markov Model of Genetic Information Encoding (1997) (Ph.D. dissertation, Georgia Institute of Technology, on file with the Georgia Institute of Technology Library, which is herein incorporated by reference in its entirety).???

Conventional programs using inhomogeneous Markov models, however, are limited to a defined probabilistic model for determining probability, and cannot be tailored by the investigator to better suit the nucleic acid sequence under study if information about that nucleic acid sequence is already available. Further, conventional implementations do not allow for the efficient and accurate detection of other nucleic acid sequence features.

### SUMMARY OF THE INVENTION

Accordingly the present invention relates to a method gene sequencing comprising:
(a) converting a DNA string to be mapped to a unique number string,
(b) eliminating open reading frame bias to generate a signal;
(c) calculating the fractal dimensions of this signal;
(d) separating the sets into coding and non-coding sets at definite pre-determined cut off values.

In one embodiment of the invention, the signal is unidimensional.

In another embodiment of the invention, a triplet ACG is present at the beginning of the sequence, A is converted into a numerical value by considering the full triplet and obtaining the value of is obtained as suffix (1,3,0) following the formula given below
V_{A}¹=1*4*4+3*4+0*1=28

where V_{A}¹ Denotes the value of A at the position 1.

In another embodiment of the invention, the window is then slid one nucleotide at a time to allow the embedded patterns in the data to be recognized.

In another embodiment of the invention, the coding and non-coding sequences are separated by
(a) converting the DNA sequence into string of number [GNS DNA using a one dimensional mapping function comprising F (x,y,z)=X*4*4+y*4+z+G; x, y,z εS, GεCn, Where G is constant. Cn set of complex number in N dimension. S={0,1,2,3}.
(b) moving the window by one base, whereby the GNS DNA is equal to one GNS signal;
(c) processing the signal using any conventional signal processing means to determine the variation or extract the biological information;
(d) calculating the fractal dimensions of the signal; and separating the sets into coding and non-coding sequences at a pre-determined cut off.

In another embodiment of the invention, the organism is a prokaryote or a eukaryote.

### DETAILED DESCRIPTION OF THE INVENTION

The present invention is in the field of bioinformatics, particularly as it pertains to gene prediction. More specifically, the invention relates to the probabilistic analysis of nucleic acid sequences for the determination of coding features, including determination of state probabilities for each nucleotide in a nucleic acid sequence, determination of coding strand, determination of open reading frame extent, determination of insertion and deletion location, determination of exon location, and determination of protein sequence.

Prior art techniques for sequencing long stretches of genomic deoxyribonucleic acid (DNA) such as cDNA hybridization, are effective at locating transcribed genes, but are time-consuming and costly, thereby increasing the cost and time for development of new drug. Statistical detection methods for locating genes on DNA that has not otherwise been analyzed for potential coding regions include using probability models to predict where in a DNA sequence a gene is located. The theoretical nucleic acid sequence probabilities can be determined through analysis of known coding regions in the organism of interest. Once theoretical nucleic acid sequence probabilities are determined, nucleic acid sequences in non-annotated regions of DNA in the same or a similar organism can be statistically compared to the theoretical nucleic acid sequence probabilities. If the similarity is sufficient, the investigator is notified that a coding sequence exists. Conventional cloning techniques can then be used to isolate the putative gene and check for transcription.

In the content based statistical detection method for searching DNA, highly conserved regions of DNA common to all genes are located. If a conserved region of DNA is found, then the nucleic acid sequence associated with the conserved region is compared with known genes. Such comparisons, which can be done with conventional nucleic acid sequence comparison programs works only if similar nucleotide or protein sequence is present and are therefore, of limited use.

The signal based statistical detection method of searching DNA involves using probability models to predict whether DNA fragments within a larger nucleic acid sequence are coding. Early searching by signal programs, such as Test Code and Grail, relied on statistical variations within coding regions of DNA, including codon frequency, local nucleic acid sequence composition, codon preference measures, heuristics based on oligonucleotide frequency variations, and measures of nucleic acid sequence complexity.

Other conventional programs for determination of coding features such as the location of intron/exon boundaries in eukaryotic organisms and the location of insertions or deletions depends on non-local nucleic acid sequence characteristics, which make the program very sensitive to sequencing errors and genes containing alternative splicing strategies.

The method of the invention essentially resides in a genomic number system. A genome is simply string of four nucleotide bases. A, T, G, C. The method of the invention comprises a number system of the base 4. Thus, the system has four digits 0,1,2,3. These numbers are assigned to the four bases according to the decreasing molecular weight as shown below:

C=3

T=2

A=1

G=0

Purine bases (G, A) assigned values 0 and 1 respectively and pyrimidine bases (T, C) given values 2 and 3.

The method of the invention is based primarily on the fact that DNA is double stranded. Both the strands carry the same information and are complementary to each other. In DNA structure the complementary pairing observed is GC and AT. When the values of GC and AT are added, a constant value of three is obtained (0+3 and 1+2=3). This is taken as the maximum number of in the number system of the invention.

This property is reflected into the signal generated by the GNS DNA. The signal generated by the DNA remains the same to its reverse, complementary and reverse complementary sequence.

In the method of the invention, accelerated processing of the DNA string is effected. Conventional gene finding algorithms process both the strands of DNA since the gene can be present on any of the strands. The conventional method adopted by any algorithm is to take the sequence and run the algorithm and then take the reverse complementary of the sequence and run the algorithm again in order to predict the genes.

As will be appreciated even simple gene prediction algorithm such as the ORF finder require at least six runs through the sequence—three times to find the positive frames [+1+2+3] and three times on the reverse complementary sequence in order to find the negative frames [−1−2−3]. Thus it is clear that the processing as well as the no of false positives are higher.

In nature both the strands carry same information so they must produce a similar signal which is recognized by the enzymes involved in the process of transcription. The method of the invention analyses the DNA sequence only once since the signal produced by both the strands is same.

The numbering system of the invention comprises of the following steps:

1. Take the Sequence and Run the Algorithm

As the signal generated by GNS DNA [The number string generated by converting the DNA using GNS] is same for DNA sequence and it's complementary sequence. This can be verified by possessing it by various signal processing techniques like Wavelet fractal ect. The fractal dimension of the GNS DNA and its Complementary GNS DNA is same.

This makes the analysis of the invention faster and unique than normal algorithms since a universal signal is captured and processed.

A. Mapping Function

The DNA string is mapped to convert it to a unique number string. A window size of three nucleotides to convert a particular nucleotide is taken and the window is slid to eliminate any ORF (open reading frame) related bias.
F(Xn, Yn, Zn)=4*4*Xn+4*Yn+Zn+Gn;
where Gn is a constant
Where GnεCn
where Cn is the set of Complex number in N dimension

These element Xn, Yn, Zn are elements of vector space in N dimension where each element can be written as linear combination of the is Basis Element.
{e1,e2,e3 . . . en);
such that
Xn=a1*e1+a2*e2+ . . . an*en.
Yn=b1*e1+b2*e2+ . . . bn*en.
Zn=c1*e1+c2*e2+ . . . cn*en.

This function give the unique number of the base placed on position Xn and having neighboring bases Yn, Zn.

Where the signal is one dimensional, such as for example where at the beginning of the sequence a triplet ACG is present, then in order to convert A into numerical value the full triplet is considered and the value of A is obtained as suffix (1,3,0) following the formula given below

Number in the GNS.
V_{A}¹=1*4*4+3*4+0*1=28

where V_{A}¹ Denotes the value of A at the position 1. In the method of this invention, the algorithm requires that not only the nucleotide but its location and local interactions (i.e. correlation between neighboring nucleotide) also be considered. The window is then slid one nucleotide at a time. This allows the embedded patterns in the data to be recognized. This technique captures the dynamics of how individual bases position related to the position of every base in the sequences.

Identification of Coding and Non Coding

The system of the invention can be extended to separate coding and non-coding sequences. Given a set of sequences the system of the invention can classify it in to protein coding or RNA producing genes and non-coding sequence.

The protocol followed is as follows:
1. The DNA sequence is converted into string of number [GNS DNA]. The one dimensional mapping function is F (x,y,z)=X*4*4+y*4+z+G x, y,z εS, GεCn, Where G is constant. Cn set of complex number in N dimension. S={0,1,2,3}.
2. Then the window is moved by one base.
3. This GNS DNA is now equivalent to the GNS Signal. Any conventional signal processing function can now be used to determine the variation or extract the biological information.
4. The Fractal Dimensions of this signal is calculated.
5. At definite cut off the sets are separated into coding and non-coding sets with high accuracy compared to the existing systems or algorithms.

The method of the invention has tremendous application in the area of bioinformatics such as identification of gene start, gene end, promter prediction, splice site prediction, alternate splice site prediction, prediction of mRNA, complete gene structure, gene prediction, novel amino acid determination, signal possessing of GNS DNA or GNS signal using different well known signal processing techniques like Hurst coefficient, Fractal Dimension, wavelet coefficient.

For example, in the case of amino acids, the amino acids are arranged such that a gray code pattern is obtained in the hydrogen bond of neighboring amino acid arrangement. This enables prediction of new amino acids. Again, analysis of different properties of proteins sequence converted into GNS proteins can be effected by using codon Periodic table. Genomic DNA can be analysed for faster drug development by generating leads or target enzymes or genes. The present invention is software based and can be implemented across any conventional laboratory data and signal processing systems such as those using Xeon-Intel Dual Processor, 3.1 GHz Speed Hard Disk 80 GB. The method briefly comprises:

Step 1: The DNA is converted into signal using GNS.

Step 2: Using signal processing techniques identifies GNS ORFs based on content and signals.

Step 3: Depending on the organisms (Prokaryotes and Eukaryotes), the further processing changes accordingly.

In the case of prokaryotes the signal of the content of GNS ORFs is studied and classified into coding or non-coding ORFs. Coding GNS ORFs are termed as GNS predicted genes. The GNS predicted genes are mapped on the main sequence result in promoter extraction. The promoter region is converted into signal processing in order to find the Transcription factor/RBS that will help converting about the regulation and expression of the predicted genes. This methodology enables determination of gene network across all the set of the genes of an organism, clustering on the basis of expression which is of immense importance in the area of the system biology. The complete gene structure is verified by collecting the data generated thereby leading to prediction of GNS mRNA and protein-protein comparisons between host and the parasites using standard algorithms or using new periodic table of codons generates leads or targets for drug discovery. In the case of eukaryotes, the GNS predicted ORFs are subjected to detect coding stretches. The GNS ORFs which shows one or more coding stretches are further analysed for detection of intron/exon boundaries. For alternate splicing all possible combinations of splicing are generated and using signal processing the right combinations are filtered/detected. The promoter region is converted into signal processing in order to find Transcription Factors that will help connecting about the regulation and expression of the predicted genes. These studies when conducted across all the set of the genes of an organisms helps to find the gene network, clustering on the basis of expression which is of immense importance in the area of the system biology. The complete genes structure are verified by collating the information leading to prediction of GNS mRNA and further protein-protein comparisons between host and the parasites using the standard algorithms or using new periodic table of codons generates leads or targets for drug discovery.

New periodic table is generated by first taking the basic table and transforming it by integer division by 4, Mode 4 and hydrogen bond pattern of amino acids. Periodic table of codon: The GNS is further extended to novel Periodic Table of Codons, which is given below. The values of codon each are calculated using Genomic Number System. They are C=3, U=2, A=1, G=0. CCC=3*4²+3*4+3=63.

TABLE 1
The Basic Table

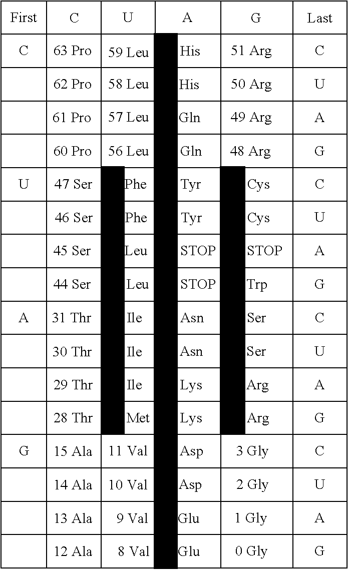

The shaded numbers separates the amino acids into two groups of amino acids which has four codon and amino acids which has two codons each.

This table has many unique properties.

1. The arrangement of codons reveals that the amino acids like Leu, Ser, Arg which has six codons in normal conventional codon table. Our classification of divides the six codons of these amino acids into two groups of four in one block and two into other block. To support this reference which discloses the different form of Leu.( Symmetry scheme for amino acid codons

J. Balakrishnan*

CSIR Centre for Mathematical Modelling and Computer Simulation (C-MMA CS), NAL Wind Tunnel Road, Bangalore-560 037, India

˜Received 30 Jul. 2001; published 25 Jan. 2002!

) stop codon some times getting translated into trp. 1981 NAR vol 15

The alternate start of gene is codon AUA is also met by the classification system using the GNS system of the invention. In the case of bacterial genetic code the alternate all start the alternate start are

TTG-Leu

CTG-Leu

ATC-Ile

ATT-Ile

ATA-Ile

ATG-Met

GTG-Val

All these also fall in the same column 2 when we transform the basic table into integer division by 4

A study of hydrogen bond interaction of these amino acids using the system of the invention lead to prediction of isomerism of amino acids i.e amino acids having same functional group but different orientation of hydrogen bonds imparting change in the prosperities, which in turn affect the functionality. This is evident from the hydrogen bond studies and the periodicity observed in table 2 below.

TABLE 2
The Basic Table Transformed using Integer Division 4

| First | C | U | A | G | Last |
|---|---|---|---|---|---|
| C | 3 | 2 | 1 | 0 | C |
| | 3 | 2 | 1 | 0 | U |
| | 3 | 2 | 1 | 0 | A |
| | 3 | 2 | 1 | 0 | G |
| U | 3 | 2 | 1 | 0 | C |
| | 3 | 2 | 1 | 0 | U |
| | 3 | 2 | 1 | 0 | A |
| | 3 | 2 | 1 | 0 | G |
| A | 3 | 2 | 1 | 0 | C |
| | 3 | 2 | 1 | 0 | U |
| | 3 | 2 | 1 | 0 | A |
| | 3 | 2 | 1 | 0 | G |
| G | 3 | 2 | 1 | 0 | C |
| | 3 | 2 | 1 | 0 | U |
| | 3 | 2 | 1 | 0 | A |
| | 3 | 2 | 1 | 0 | G |

An analysis of the basic table transformed using integer division by four shows that the table can be divided into four columns—0,1,2, and 3—the basic GNS numbers. The list of elements in each columns is an exact match of mutation ring of Siemion et al 1992 1994a. list, which also proposes that the classification of amino acids be on the basis of their various properties.

FIG. 5 is a Siemon mutation ring. An analysis of this figure shows that the first two positions in codon governs the chemical nature or properties of amino acids coded by it. In other words the last digit does not play an important role in determining the properties of the amino acids coded by it. In the system of the invention two groups of the generated table data four codons code for single amino acids are observed. In the case of second group which is inside two codons code for single amino acids. The column wise arrangement of amino acids they exhibit similar properties.

Table 3 below shows the generation of a number to amino acids which can be derived from the transformed tables [Integer Division four and Mode four] which can substitute it in the GNS proteins for further studies of protein—protein interactions comparison.

TABLE 3
The Basic Table Transformed using MOD 4

| First | C | U | A | G | Last |
|---|---|---|---|---|---|
| C | 3 | 3 | 3 | 3 | C |
| | 2 | 2 | 2 | 2 | U |
| | 1 | 1 | 1 | 1 | A |
| | 0 | 0 | 0 | 0 | G |
| U | 3 | 3 | 3 | 3 | C |
| | 2 | 2 | 2 | 2 | U |
| | 1 | 1 | 1 | 1 | A |
| | 0 | 0 | 0 | 0 | G |
| A | 3 | 3 | 3 | 3 | C |
| | 2 | 2 | 2 | 2 | U |
| | 1 | 1 | 1 | 1 | A |
| | 0 | 0 | 0 | 0 | G |
| G | 3 | 3 | 3 | 3 | C |
| | 2 | 2 | 2 | 2 | U |
| | 1 | 1 | 1 | 1 | A |
| | 0 | 0 | 0 | 0 | G |

Hydrogen Bond Periodicity of New Periodic Table

Hydrogen bond provides conformation of protein molecules. The protein chain is modeled by an n-arc graph with the following elements, vertices (α carbon atom), structural edges (peptides bonds) and connectivity edges (virtual edges connecting non-adjacent atoms).

The capacity of the main and side chains of chained polymers to fix the conformation of the latter was assumed as a prerequisite of their self-assembly. (Karasev et al, 2000). Such a capacity was called connectivity. Polypeptides are chained polymers possessing connectivity. Their conformation is fixed due to hydrogen bond and their interaction.

The polypeptide chain can be represented by the n-arc graph. The 4-arc graph is a minimal model. Vertices (I,I-1, . . . , I-4) correspond to the α-carbon atom of the periodically repeated unit (residues) of protein molecule. Structural edges Ks (solid line), connectivity vicinal vertices, represent corresponding peptide bonds. To model fragment of the protein molecule fixed due to hydrogen bond or otherwise, we close the graph with a “connectivity (virtual) edge. If the occurrence of a connectivity edge is denoted as “1” and the absence of such an edge as “0”. The general form of the matrix, describing the connectivity state of the 4-arc graph is shown in FIG. 6.

### EXAMPLE 1

Comparison of conventional GeneScan and the system of the invention on a common data set “HMR 195”: Reference: (Rogic et al., 2001) Sanja Rogic, Computer Science Department 2366 Main Mall, University of British Columbia, Vancouver, B.C., Canada V6T 1Z4 11

DNA sequences were extracted from GenBank. The basic requirements in sequence selection were that the sequence was entered in GenBank after August, 1997 and the source organism is Homo sapiens, Mus musculus or Rattus norvegicus. Only genomic sequences that contain exactly one gene were considered. mRNA sequences and sequences containing pseudo genes or alternatively spliced genes were excluded. Sequences collected according to those principles were further filtered to meet following requirements. All annotated coding sequences started with the ATG initiation codon and ended with one of the stop codons: TAA. TAG, TGA. All exons had dinucleotide AG at their acceptor site and dinucleotide GT at their donor site. Sequences that did not contain any nucleotides in their 5′ or 3′ UTR were discarded. Sequences longer than 200,000 bp were discarded because some of the programs analyzed can only accept sequences up to that length. Sequences whose coding region contains in-frame stop codon were discarded HMR195 has the following characteristics:
The ratio of Human:Mouse:Rat sequences is 103:82:10
The mean length of the sequences in the set is 7,096 bp
The number of single-exon genes is 43 and the number of multi-exon genes is 152
The average number of exons per gene is 4.86
The mean exon length is 208 bp, the mean intron length is 678 bp and the mean coding length of a gene is 1,015 bp (˜330 amino acids)

The proportion of coding sequence in this dataset is 14%, of the intronic sequence 46% and of the intergenic DNA 40%. The Analysis was carried out after separating the introns and exons from the dataset. Each file was parsed and separate Intron and Exon sequences were generated. These sequences were subjected to both the methods.

Genescan: This is an ab initio method which converts the DNA sequence into Power Spectrum and then calculates the frequency at 1/3. Exploits the property of Coding DNA sequence that it follows 3 base periodicity.[Tiwari et al 1997]. Study on different dataseta have shown that the threshold value which separates the coding and noncoding sequences i.e. Exon and Intron respectively is around 4.00

GNS: the method of the invention was used to convert the DNA sequence into a signal. The fractal dimension of the signal is calculated and the data generated used to calculate the sensitivity and specificity on the new algorithm at different cutoff ranging from 0.75 to 1.25 [FIG. 1].

A sensitivity and specificity analysis of the system of the invention establishes that it is essential to balance between both sensitivity and specificity. The optimal threshold value which separates the positive and the negative sets is equal to “0.9172” and the sensitivity and specificity achieved at this threshold is 0.859600 and 0.715789 respectively for the HMR 195 data set. The data generated by running both GeneScan and the system of the invention are given in Table 1 below.

Table: 2 Comparative Study Table (Cutoff Used in System of Invention=0.9172)

In Row Nos. 7, 24, and 28, it was observed that the Genescan provided a better performance than the method of the invention.

In Row Nos. 12-15, 19-20 and 25-27, it was observed that the method of the invention is comparable to Genescan.

In all remaining rows, the results of the system of the invention were significantly higher than those of Genescan.

| | | | Gene Scan | | | GNS Method | | |
|---|---|---|---|---|---|---|---|---|
| | Total | Total | Exon False | Intron False | | Exon False | Intron False | |
| S. No | Exon | Intron | Negative | Negative | Percentage | Negative | Negative | Percentage |
| 1 | 10 | 9 | 2 | 1 | 80 | 0 | 0 | 100 |
| 2 | 1 | 0 | 0 | 0 | 100 | 0 | 0 | 100 |
| 3 | 2 | 1 | 0 | 0 | 100 | 0 | 0 | 100 |
| 4 | 10 | 9 | 4 | 2 | 60 | 3 | 4 | 70 |
| 5 | 5 | 4 | 2 | 0 | 60 | 1 | 2 | 80 |
| 6 | 18 | 17 | 7 | 0 | 61.11111 | 3 | 4 | 83.33 |
| 7 | 7 | 6 | 1 | 1 | 85.71429 | 2 | 3 | 71.42857 |
| 8 | 4 | 3 | 1 | 0 | 75 | 0 | 0 | 100 |
| 9 | 7 | 6 | 1 | 2 | 85.71429 | 1 | 1 | 85.71 |
| 10 | 13 | 12 | 9 | 0 | 30.76923 | 2 | 5 | 84.61 |
| 11 | 11 | 10 | 1 | 2 | 90.90909 | 0 | 3 | 100 |
| 12 | 3 | 2 | 1 | 0 | 66.66667 | 1 | 0 | 66.66667 |
| 13 | 3 | 2 | 1 | 0 | 66.66667 | 1 | 0 | 66.66667 |
| 14 | 1 | 0 | 0 | 0 | 100 | 0 | 0 | 100 |
| 15 | 1 | 0 | 0 | 0 | 100 | 0 | 0 | 100 |
| 16 | 14 | 13 | 6 | 1 | 57.14286 | 3 | 0 | 78.57 |
| 17 | 4 | 3 | 3 | 1 | 25 | 1 | 0 | 75 |
| 18 | 28 | 27 | 8 | 5 | 71.42857 | 2 | 11 | 92.85 |
| 19 | 1 | 0 | 0 | 0 | 100 | 0 | 0 | 100 |
| 20 | 3 | 2 | 0 | 1 | 100 | 0 | 0 | 100 |
| 21 | 3 | 2 | 3 | 0 | 0 | 1 | 0 | 66.66667 |
| 22 | 3 | 2 | 1 | 0 | 66.66667 | 0 | 0 | 100 |
| 23 | 7 | 6 | 3 | 0 | 57.14286 | 1 | 3 | 85.71 |
| 24 | 6 | 5 | 0 | 1 | 100 | 1 | 2 | 83.33333 |
| 25 | 1 | 0 | 0 | 0 | 100 | 0 | 0 | 100 |
| 26 | 2 | 1 | 0 | 0 | 100 | 0 | 0 | 100 |
| 27 | 1 | 0 | 0 | 0 | 100 | 0 | 0 | 100 |
| 28 | 2 | 1 | 0 | 0 | 100 | 1 | 0 | 50 |
| 29 | 171 | 143 | 54 | 17 | 68.42105 | 24 | 38 | 85.9649 |

1-7. (canceled)

8. A method for gene sequencing comprising:
(a) converting a DNA string to be mapped to a unique number string;
(b) eliminating open reading frame bias to generate a signal;
(c) calculating the fractal dimensions of this signal; and
(d) separating the sets into coding and non-coding sets at definite pre-determined cut off values.

9. The method as claimed in claim 8, wherein the signal is unidimensional.

10. The method as claimed in claim 9, wherein a triplet ACG is present at the beginning of the sequence, A is converted into a numerical value by considering the full triplet and obtaining the value is obtained as suffix (1,3,0) following the formula given below
V_{A}¹=1*4*4+3*4+0*1=28
where V_{A}¹ denotes the value of A at position 1.

11. The method as claimed in claim 8, further comprising sliding a window one nucleotide at a time to allow the embedded patterns in the data to be recognized.

12. The method as claimed in claim 8, wherein the coding and non-coding sequences are separated by
(a) converting the DNA sequence into string of numbers [GNS DNA using a one dimensional mapping function comprising F (x,y,z)=X*4*4+y*4+z+G; x,y,z εS, Gε=Cn, where G is constant. Cn set of complex number in N dimension. S={0,1,2,3};
(b) moving the window by one base, whereby the GNS DNA is equal to one combined single GNS signal;
(c) processing the signal using any conventional signal processing means to determine the variation or extracting the biological information; and
(d) calculating the fractal dimensions of the signal and separating the sets into coding and non-coding sequence at a pre-determined cut off.

13. The method as claimed in claim 12, wherein the organism is a prokaryote.

14. The method as claimed in claim 12, wherein the organism is a eukaryote.
